(19) United States
(12) Patent Application Publication
(10) Pub. No.: US 2026/0207824 A1
(43) Pub. Date: Jul. 23, 2026

(54) GALLIUM-LOADED HYDROGELS FOR BONE TREATMENT

(71) Applicant: NORTHEAST OHIO MEDICAL UNIVERSITY, Rootstown, OH (US)

(72) Inventors: Moses OYEWUMI, Rootstown, OH (US); Pratyusha GHANTA, Rootstown, OH (US)

(21) Appl. No.: 19/141,168

(22) PCT Filed: Dec. 20, 2023
(86) PCT No.: PCT/US2023/085203

§ 371 Date: Jun. 19, 2025

Related U.S. Application Data
(60) Provisional application No. 63/433,882, filed on Dec. 20, 2022.
(60) Provisional application No. 63/465,153, filed on May 9, 2023.

(51) Int. Cl. A61L 27/54; A61L 27/20; A61L 27/26; A61L 27/52; A61L 27/58; C08L 1/02; C08L 1/28; C08L 67/04; C08L 71/02

(52) U.S. Cl. CPC A61L 27/54; A61L 27/20; A61L 27/26; A61L 27/52; A61L 27/58; C08L 1/02; C08L 1/28; C08L 67/04; C08L 71/02; A61L 2300/224; A61L 2430/02

(57) ABSTRACT

A composition for inhibiting bone resorption including a gallium compound and a thermoresponsive hydrogel is described. Methods of inhibiting bone resorption in a subject by administering a therapeutically effective amount of composition including a gallium compound and a thermoresponsive hydrogel are also described.

### CROSS-REFERENCE TO RELATED APPLICATIONS

This application claims priority to U.S. Provisional Patent Application Ser. No. 63/433,882, filed Dec. 20, 2022, and U.S. Provisional Patent Application Ser. No. 63/465,153, filed May 9, 2023, the disclosures of which are incorporated herein by reference.

### BACKGROUND

Bone is a dynamic and mineralized connective tissue that functions through a cyclic homeostatic process of remodeling and resorption, sustained throughout life. Osteoblasts (OBs) are a category of bone cells that are responsible for secreting organic matrix and deposition of minerals to create new bone. In contrast, osteoclasts (OCs), multinucleated bone cells derived from hematopoietic precursors, are directly involved in the resorption of the bone matrix. The functioning of these bone cells is convoluted where OBs secrete cytokines like macrophage colony-stimulating factor (M-CSF) and receptor activator of NFKB ligand (RANKL) that stimulate osteoclast differentiation and function during the active remodeling phase of bone regeneration. Essentially, potential disruption in either bone cells'functioning will lead to an imbalance in bone homeostasis. Such homeostatic imbalance can lead to skeletal fragility or abnormal bone formation depending on whether OC or OB functions are enhanced or suppressed.

Specifically, OCs are differentiated multinucleated cells from mononuclear cells of the hematopoietic stem cell lineage. The differentiation process occurs in the presence of many factors, mainly macrophage colony-stimulating factors (M-CSF), secreted by osteoprogenitor mesenchymal cells; and receptor activation of nuclear factor kappa-B ligand (RANKL), which is secreted by OBs, osteocytes (OCTs), and stroma cells. RANKL/RANK interaction promotes the expression of the following osteoclastogenic factors: TRAF6, nuclear factor of activated T cells 2(NFAT2 ), DC-STAMP, and OC STAMP. Osteoclastogenesis (production of OCs) has been reported to be regulated through the pathways involving NF-κB, cFos, JNK, etc. Upon complementary binding of RANKL to RANK receptor, TRAF6 is activated via phosphorylation, and in turn, transcription factors such as cFos and NFAT2 are stimulated. cFos is present in the initial stages of the OC differentiation and later regulates the autoamplification of NFAT2 and other OC markers. Mature OCs bind tightly to the bone and create a sealed microenvironment where they produce collagenolytic enzymes like cathepsin k (CTSK) and other secreted protons that affect the organic matrix and degrade the mineral component. Interestingly, OBs, OCT, and stromal cells secrete factors to stimulate osteoclastogenesis; however, osteoprotegerin (OPG), produced by the same cells, inhibits osteoclastogenesis through negative feedback.

In most bone resorptive diseases, the attributable mechanisms could result from either downregulation of OPG or upregulation of RANKL and M-CSF or other factors that contribute to the imbalance in the bone homeostasis. Potential disruption of the function of OC or OB will interfere with the process of bone remodeling, which is crucial in conditions such as fractures, skeletal adaptation, and calcium homeostasis. An imbalance in this process can lead to severe bone-related disorders. For instance, excessive resorption by overstimulated OCs can contribute to excessive bone loss, resulting in disorders such as osteoporosis and Paget's disease. The impact of overstimulated osteoclastic bone resorption is associated with skeletal fragility and a high propensity for fracture, notable features of osteoporosis. Osteoporosis heavily contributes to the 1.5 million fractures that occur yearly in the United States.

In this regard, therapeutic agents have been developed for the management of osteoporosis. Similar diseases target the inhibition of relevant pathways and proteins associated with osteoclastic bone resorption. However, current therapy generally only includes the prevention of fracture after the initial fracture occurs. This lapse in therapy can be explained by the limitations of the current products in the market. Schlickewei et al., Int J Mol Sci, 20(22), 5805(2019 ). Preventative agents are those indicated for osteoporosis, though some of which can aid in the regeneration of bone. Examples include agents like Forteo®, and hormone replacement therapy with estrogen, calcitonin, and bisphosphonates. However, there are limitations surrounding the routine use of these products, such as high cost, poor compliance, risks, and side effects, mixed results, and poor pharmacokinetic profile without other modulating agents. Lee et al., Sci Rep, 6: p. 27336(2016 ) Additionally, there are approved agents used specifically for bone regeneration, for example, osteoinductive agents and bone morphogenic proteins 2 and 7 (BMP-2 and BMP-7). The effectiveness of these agents has been plagued by extensive adverse events such as post-operative inflammation and adipogenesis. James et al., Tissue Eng Part B Rev, 22(4): p. 284-97 (2016) There is a need for effective therapeutic agents to manage osteoclastic bone resorption while maintaining attractive safety profiles.

### SUMMARY OF THE INVENTION

Gallium compounds have gained much attention for their therapeutic and diagnostic properties. Verron et al., Drug Discov Today, 17(19-20): p. 1127-32 (2012). Notably, gallium compounds are effective against different types of cancers. Warrell et al., Cancer Res, 46(8): p. 4208-12 (1986). Gallium nitrate is approved for treating malignancy-associated hypercalcemia. Warrell et al., Cancer research, 46(8): p. 4208-4212 (1986). Further, gallium nitrate has been shown in multiple studies to inhibit osteoclastogenesis (Verron et al., Br J Pharmacol, 159(8): p. 1681-92 (2010)); the trend has been substantiated with inhibition of various signaling pathways responsible for OC differentiation and bone resorption such as Tartrate resistant acid phosphatase (TRAP), cathepsin K (CTSK), RANK, OC-Stamp, NFAT2, NFKB, calcitonin receptor (CTR), and transient receptor potential cation channel subfamily V (TRPV5).

The inventors believe that the gallium compound holds great potential as a safe and effective treatment option for osteoclastic bone resorption and is worthy of further investigation. Meanwhile, the potential application of gallium compounds will require suitable delivery systems that enhance retention at the bone site and decrease off-target distribution while enhancing efficacy. They are not aware of previous work on delivery systems for the application of gallium compounds in bone resorption. Thus, the studies described herein investigate the efficacy of a new gallium compound, gallium acetylacetonate (GaAcAc), against osteoclastic bone resorption and to fabricate and assess the effectiveness of delivery systems using both in-vitro and in-vivo approaches.

### BRIEF DESCRIPTION OF THE FIGURES

The present invention may be more readily understood by reference to the following figures, wherein:

FIGS. 1A and 1B provide graphs showing the in-vitro biocompatibility of GaAcAc. Various concentrations of GaAcAc were incubated (37° C.) with (A) pre-OC cells (RAW 264.7) or (B) pre-OB cells (MC3T3-E1). Percent cell viability was assessed using untreated control cells as a reference. Each data point represents mean±SD (n=6); #p>0.05.

FIGS. 2A-2C provide graphs and images showing the effects of GaAcAc on OC Differentiation: Pre-osteoclastic (RAW 264.7) cells were cultured and differentiated into OC with growth media containing RANKL (30 ng/ml)(positive control). Cells were treated with GaAcAc solution at 10 & 50 μg/mL together with RANKL (30 ng/ml). The extent of OC differentiation was assessed by (A) TRAP activity, (B) the number of multi-nucleated cells (OC) with TRAP-stained containing 3 or more nuclei, and (C) the extent of TRAP staining (white arrows). Each data point represents mean±SD; n=6. *p<0.05, * p<0.001 and ***p<0.0001 vs positive control (RANKL).

FIGS. 3A-3D provide graphs and images showing the development and characterization of methylcellulose hydrogels (MH): (A-B) Rheological characterization of MH prepared at 6 & 8% w/v of methylcellulose based on storage modulus (G′ as the red line) and loss of modulus (G″ as the blue line) vs Temperature (C). (C) Representative photographs of thermogelling behavior of MH (8% w/v methylcellulose) at 4° C., 25° C., and 37° C. (D) In-vitro degradation of hydrogel layer as measured by percentage retention of MH layer when stored with PBS on top of the gel. Each data point represents mean±SD; n=3 separate formulations. **p<0.001 and ***p<0.0001.

FIGS. 4A & 4B provide graphs showing the characterization of GaMH (GaAcAc loaded Methylcellulose Hydrogels): (A) Effects of various concentrations of GaAcAc on gelation temperatures of GaMH as obtained from rheological measurements. (B) In-vitro release profile of GaAcAc from GaMH. Each data point represents mean±SD; n=3-4, #p>0.05, *p<0.05 and ***p<0.0001 vs positive control (blank hydrogels).

FIGS. 5A-5C provide graphs and images showing the effects of GaMH on OC Differentiation. Murine hematopoietic stem cells were collected and differentiated to OCs with different treatments of RANKL and M-CSF alone (positive control) or together with MH or GaMH (containing GaAcAc 10 μg/mL). At the end of differentiation, cells were assessed for (A) TRAP activity, (B) number of multi-nucleated cells (OC) with TRAP-stained containing with 3 or more nuclei. Only cells with 3 or more nuclei and TRAP stained, i.e., pink color, were counted, and (C) extent of TRAP staining. Each data point represents mean±SD; n=3, #p>0.05 and ***p<0.0001 versus positive control (RANKL).

FIGS. 6A & 6B provide images showing the mechanistic assessment of OC Differentiation Markers. Expression of key markers of OC differentiation as analyzed by western blotting (A) cFos as well as (B) NFAT2, TRAF6, and TRAP. RAW 264.7 cells were differentiated into OCs with treatment with RANKL alone (control) or RANKL together with any of the following: GaAcAc solution (10 μg/mL and 50 μg/mL), blank hydrogel (MH), and GaMH (loaded with 10 μg/mL).

FIGS. 7A-7C provide graphs and images showing the ex-vivo characterization of Osteoclastic Bone Resorption after GaMH Treatment. RAW 264.7 cells were differentiated to OCs on bovine cortical slices with RANKL treatment alone (positive control) or RANKL treatment together with any of the following: GaAcAc solution (10 μg/mL) or blank hydrogel (MH) or GaMH (containing 10 μg/mL GaAcAc). After OC differentiation process, the bone slices were stained via (A) TRAP for the presence of differentiated OCs (white arrows) and (B) stained with 0.5% toluidine blue to count for osteoclastic bone resorbed pits on bone slices after various treatments. (C) CTSK analysis by ELISA of cell media during OC differentiation on bone slices. Each data point represents mean±SD; n=3. **p<0.001, ***p<0.0001 vs positive control (RANKL).

FIGS. 8A & 8B provide graphs and images showing the in-vivo bio-retention of GaMH after injection in BALB/c mice. (A) Representative IVIS images of BALB/c mice after injection of fluorescent-labeled GaMH (GaMH-ICG) or ICG solutions into the lower right hindlimb (periosseous) of BALB/c mice. Animals in all groups were imaged at different time intervals after injection. (B) Concentration of gallium (Ga) retained at injection site in BALB/c mice. Mice were injected with GaAcAc solution or GaMH as a single perisseous injection into the lower right hindlimb of BALB/c mice. The extent of Ga retention in each mouse (2 hrs and 24 hrs post injection) was expressed as a ratio of concentration of Ga at the injection site (Inj. Site) versus a non-injection site (Non-Inj Site) when measured by ICP. Each data point represents mean±SD; n=3 mice (*p<0.05).

FIGS. 9A & 9B provide graphs and images showing solutions of GaAcAc injected into mice did not show detectable toxicity based on weight of animals. A solution of GaAcAc was injected into BlkC57 mice. Groups included in the study are: control: No injections; 1×/week: 100 μL of 100 μg/mL Ga solution injected subcutaneously in the back once per week for 4 weeks; 2×/week: 100 μL of 100 μg/mL Ga solution injected subcutaneously in the back twice per week for 4 weeks; 3×/week: 100 μL of 100 μg/mL Ga solution injected subcutaneously in the back three times per week for 4 weeks.

FIGS. 10A-10D provide graphs and images showing characterization of the thermoresponsive behavior of the PLA-b-PEG-b-PLA hydrogel: (A) Observational vial inversion studies-Photographs of the thermogelling behavior of the 10% (w/v), 20% (w/v), and 30% (w/v) copolymeric hydrogels at varying temperatures; (B) Laser light scattering measurements-Representative particle size distribution of 10% (w/v), 20% (w/v), and 30% (w/v) copolymeric hydrogels. The particle size distribution data were obtained at varying temperature conditions; (C) and (D) Rheological Profiles of the Copolymeric Hydrogels-Representative rheological behavior profiles of 10% (w/v) and 20% (w/v) copolymeric hydrogels over 20-45° C., reported as storage (G′) (blue) and loss (G″) (green) moduli for each formulation. Phase transition values were 34° C., and 33° C. for the 10% (w/v) and 20% (w/v) copolymeric hydrogels, respectively.

FIGS. 11A-11D provide graphs showing characterization of polymersomes Ga-Ps. Polymersomes loaded with Ga (0-100 μg/ml) were prepared and characterized based on (A) size and (B) zeta potential measured by DLS on day 0 (day of preparation) as well as by (C) entrapment efficiency (EE%) upon loading of various concentrations of Ga and (D) size stability in water, PBS or PBS containing 10% (v/v) FBS from day 0 thru day 7 after preparation at 37° C. Data points are plotted as average +SD (n=4 separate formulations). #represents (p>0.05), *** represents (p<0.0001).

FIG. 12 provide a graph showing in-vitro release of Ga from polymersomes Ga-Ps in PBS. Cumulative % release of Ga from Ga-Ps (100 μg/ml) as compared to Ga alone (GaAcAc) (mean ±SD; n=4 separate formulations). At specific time intervals, 8 ml of the release medium was collected and analyzed for Ga.

FIGS. 13A & 13B provide graphs showing leachates from hydrogels prepared using Pluronic F127 did not affect osteoclast differentiation. Pre-osteoclast (RAW 264.7) cells were differentiated by treating the cells with culture media supplemented with or without different concentration of F127 hydrogel (HG) leachates and 30 ng/ml RANKL. The cells were then assessed for TRAP activity and stained. (A) TRAP activity; (B) OC count. Only cells which have 3 or more nuclei and were TRAP stained, i.e., pink color were counted. Each data point represents Mean±SD; n=4 wells. *** p<0.0001, *p<0.01 vs RANKL.

FIG. 14 provides a graph showing cumulative GaAcAc release from F127 Hydrogel. GaAcAc was loaded was in two different ways: GaAcAc (100 μg/mL) was mixed with the F127 powder prior to making it into an aqueous solution, and GaAcAc (100 μg/mL) was mixed after the preparing the F127 hydrogel.

FIG. 15 provides a graph showing cumulative GaAcAc release from different hydrogel formulations. Various formulations of different proportions were prepared with GaAcAc loaded. Cumulative GaAcAc release was assessed. The studies also showed the investigation of combinations of hydroxypropyl cellulose and F127 in hydrogel preparation.

### DETAILED DESCRIPTION OF THE INVENTION

The present invention provides a composition for inhibiting bone resorption including a gallium compound and a thermoresponsive hydrogel. Methods of inhibiting bone resorption in a subject by administering a therapeutically effective amount of composition including a gallium compound and a thermoresponsive hydrogel are also provided.

### Definitions

As used herein, the terms “treatment”, “treating”, and the like, refer to obtaining a desired pharmacologic or physiologic effect. The effect may be therapeutic in terms of a partial or complete cure for a disease or an adverse effect attributable to the disease. “Treatment”, as used herein, covers any treatment of a disease in a mammal, particularly in a human, and can include inhibiting the disease or condition, i.e., arresting its development; and relieving the disease, i.e., causing regression of the disease.

Prevention, as used herein, refers to treatment of a subject identified as being at risk of being afflicted with a condition or disease such as osteogenesis imperfecta, including avoidance of development of a bone disease or disorder, or a decrease of one or more symptoms of the bone disease or disorder should a bone disease or disorder develop nonetheless.

“Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of an agent which will achieve the goal of improvement in disease severity and the frequency of incidence. The effectiveness of treatment may be measured by evaluating a reduction in symptoms in a subject in response to contact with the gallium-including hydrogels described herein.

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human.

“Contacting,” as used herein, refers to causing two items to become physically adjacent and in contact, or placing them in an environment where such contact will occur within a short timeframe. For example, contacting a site with a composition comprising a gallium compound and a cellulose-based hydrogel includes administering the composition to s subject at or near a site such that the gallium compound will interact with the site to inhibit bone resorption. In some embodiments, the step of contacting the site comprises surgically implanting the composition.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a compound” also includes a plurality of such compounds.

### Compositions for Inhibiting Bone Resorption

In one aspect, a composition for inhibiting bone resorption, comprising a gallium compound and a thermoresponsive hydrogel is provided. Preferably, the gallium compound is mixed within the thermoresponsive hydrogel. The gallium compound may be disposed on or in the biocompatible material by methods known to those skilled in the art, including by hand, electrospraying, ionization spraying or impregnating, vibratory dispersion (including sonication), nozzle spraying, compressed-air-assisted spraying, brushing and/or pouring.

Gallium compounds, as used herein, refers to gallium and gallium-including compounds, and in particular salts and coordination complexes of gallium. Preferably, the gallium compound is a non-radioactive gallium compound. In some embodiments, the gallium compound is selected from the group consisting of gallium acetylacetonate, gallium nitrate, and gallium citrate, gallium maltolate, gallium carbonate, gallium acetate, gallium triacetate, gallium tartrate, gallium oxide, gallium hydroxide, and gallium hydrated oxide. In further embodiments, the gallium compound is gallium acetylacetonate (Ga(acac)₃) which is a coordination complex of gallium having the formula Ga(C₅H₇O₂)₃. A therapeutically effective amount of the gallium compound should be included in the composition. In some embodiments, the gallium compound composition ranges from 5 μg/mL to 100 μg/mL of gallium compound/hydrogel.

The composition for inhibiting bone resorption includes a thermoresponsive hydrogel. Thermoresponsive hydrogels are hydrogels that respond to changes in temperature by converting from solution at one temperature (e.g., room temperature) to gel at a higher temperature (e.g., body temperature). Thermoresponsive hydrogels include a variety of different types of polymers, including cellulose-based hydrogels, block polymers, and natural polymers. A variety of thermoresponsive cellulose-based hydrogels are known to those skilled in the art. Jain et al., J. Appl. Pharm. Sci., 3(12), 139-144 (2013). Examples of thermoresponsive cellulose-based hydrogels include methyl cellulose, hydroxypropyl methylcellulose, and ethyl (hydroxyethyl) cellulose. Examples of block polymer-based hydrogels include poly(ε-caprolactone-co-lactide)-b-poly-(ethylene glycol)-b-poly(ε-caprolactone-co-lactide) (PCLA-b-PEG-b-PCLA), Polylactic glycolic acid-b-polyethylene-b-Polylactic glycolic acid PLGA-PEG-PLGA, Polylactic acid-b-polyethylene-b-Polylactic acid (PLA-b-PEG-b-PLA), and Pluronic F127. Examples of natural polymer-based hydrogels include Hyaluronic acid, gelatin, chitosan, and alginate.

In some embodiments, the thermoresponsive hydrogel is a cellulose-based hydrogel. A wide variety of cellulose-based hydrogels are known to those skilled in the art. Kabir et al., Prog Biomater., 7:153-174 (2018). Generally, hydrogels based on cellulose comprising many organic biopolymers including cellulose, chitin, and chitosan, which can absorb and retain a huge proportion of water in the interstitial sites of their structures. Cellulose-based hydrogels can be prepared from pure and native cellulose by chemical dissolution with LiCl/dimethylacetamide (DMAc), N-methylmorpholine-N-oxide (NMMO), ionic liquids (ILs), alkali/urea (or thiourea), or by fabricating/designing with bacterial cellulose. Cellulose derivatives are usually comprised of either esters (e.g., cellulose acetate (CA), cellulose acetate phthalate (CAP), cellulose acetate butyrate (CAB), cellulose acetate trimellitate (CAT), hydroxypropyl methylcellulose phthalate (HPMCP)) or ethers (e.g., methylcellulose (MC), ethyl cellulose (EC), hydroxyethyl cellulose (HEC), carboxymethyl cellulose (CMC), sodium carboxymethyl cellulose (NaCMC), hydroxypropyl cellulose (HPC) and hydroxypropyl methylcellulose (HPMC)).

In some embodiments, the thermoresponsive cellulose-based hydrogel is a methylcellulose hydrogel, which are cellulose ether derivatives. Examples of methylcellulose hydrogels include methylellulose, hydroxyethyl methylcellulose, and hydroxypropyl methylcellulose.

In some embodiments, the thermoresponsive block polymer-based hydrogel is a PLA-b-PEG-b-PLA hydrogel. In some embodiments, the thermoresponsive block polymer-based hydrogel is a Pluronic F127, also known as poloxamer 407, hydrogel. In some embodiments, the thermoresponsive hydrogel comprises a plurality of different polymers. For example, the thermoresponsive hydrogel can be a combination of a cellulose-based hydrogel and a block polymer-based hydrogel. For example, the thermoresponsive hydrogel can be a combination of a cellulose-based hydrogel and a block polymer-based hydrogel is a combination of hydroxypropyl cellulose and Pluronic F127.

In some embodiments, the composition comprising a gallium compound and a thermoresponsive hydrogel further comprises polymersomes. Polymersomes are a type of nanoparticles that are self-assembled vesicles composed of amphiphilic block copolymers. In some embodiments, the polymersomes have a size ranging from 150 nm to 300 nm, or from 200 nm to 250 nm. In some embodiments, the polymersomes are PLA-PEG copolymeric polymersomes. In some embodiments, GaAcAc is loaded onto polymertsomes.

In some embodiments, the thermoresponsive hydrogel comprises a bone-seeking ligand. A bone-seeking ligand is a compound that can be included in the hydrogel that has an affinity for bone that encourages association of the hydrogel with bone. Examples of bone seeking ligands include alendronate, polyglutamic acid, and polyaspartic acid. See Wang et al., Bioconjugate Chemistry, 14(5): 853-859 (2003). In some embodiments, the bone-seeking ligand is conjugated to the cellulose-based hydrogel.

In additional embodiments, the thermoresponsive hydrogel comprises a bone-retentive ligand. As used herein, a bone-retentive ligand refers to a material that can be conjugated and/or added to the polymers used in making hydrogels to increase retention at the bone fusion site. The bone-retentive ligand helps to increase the retention of the drug at the bone fusion site and prevent further diffusion out of site of application. Examples of bone-retentive ligands are polyaspartic acid, bisphosphonate, aspartic acid, glutamate, acidic oligopeptides, bisphosphonates, and alendronate.

### Methods for Inhibiting Bone Resorption

In another aspect, a method of inhibiting bone resorption in a subject is provided. The method includes administering a therapeutically effective amount of composition comprising a gallium compound and a thermoresponsive hydrogel to a subject in need thereof.

Bone resorption is the process by which osteoclasts break down the tissue in bone, releasing the minerals and resulting in a transfer of calcium from bone tissue to the blood. While bone resorption is generally a healthy process involved in routine bone remodelling, in some cases it can be helpful to inhibit bone resorption, resulting in a decreased rate of bone tissue breakdown.

A change in the level of bone resorption can readily be determined by comparing levels in a subject before and after treatment. The level of bone resorption and/or bone mass before and/or after treatment may be determined from a series of measurements taken over different timepoints to provide a standard range. The level of bone resorption and/or bone mass before and/or after treatment may be measured in multiple individuals to provide a standard range representative of a given population. In certain embodiments, the level of bene resorption may be decreased in a subject treated by the methods of the present invention by at least about 2%, at least about 3%, at least about 4%, at least about 5%, at least about 10%, at least about 15%, at least about 20%, at least about 30%, at least about 40%, at least about 50%, at least about 60%, at least about 70%, or at least about 80%, compared to the level of bone resorption prior to treatment.

A bone (i.e., bone tissue) is a rigid organ that constitutes part of the vertebral skeleton. Bone tissue includes two basic types-cortical (the hard, outer layer of bone) and cancellous bone (the interior trabecular or spongy bone tissue), which gives it rigidity and a coral-like three-dimensional internal structure. Other types of tissue found in bone include marrow, endosteum, periosteum, nerves, blood vessels and cartilage. Bone is an active tissue composed of different cells. Osteoblasts are involved in the creation and mineralization of bone; osteocytes and osteoclasts are involved in the reabsorption of bone tissue. The mineralized matrix of bone tissue has an organic component mainly of collagen and an inorganic component of bone mineral made up of various salts.

The present invention can be used to decrease the rate of bone resorption any type of bone. There are five types of bones in the human body. These are long bones, short bones, flat bones, irregular bones and sesmoid bones. Examples of long bones include the femur, the humerus and the tibia. Examples of short bones include carpals and tarsals in the wrist and foot. Examples of flat bones include the scapula, the sternum, the cranium, the os coxae, the pelvis, and ribs. Irregular bones are those which do not fit within the other categories, and include vertebrae, sacrum and mandible bones. Sesmoid bones are typically short or irregular bones, imbedded in a tendon, such as the patella. While not formally considered bone, teeth are also included in the definition of bone used herein.

Bone injury can occur as a result of disease, chronic stress, or physical trauma. Examples of different types of bone injury include degenerative disc, cervical spondylosis, and bone fracture. Bone regeneration is also called remodeling and occurs at the cellular level. When the process becomes unbalanced, e.g., from too much resorption, bone mass decreases and bones may become brittle. Decreasing the rate of bone resorption that occurs over a given time can be used to increase bone repair. For example, enhancing bone repair includes decreasing the rate or amount of bone resorption by up to 10%, 20%, 30%, 40%, 50%, 60%, 70%, 80%, 90%, or even 100% compared with the amount or rate of bone resorption that would occur in an untreated subject.

In some embodiments, the subject has been diagnosed as having a bone growth disease or disorder, or suspected to be suffering from a bone growth disease or disorder. Alternatively, subjects treated may not be suffering from a bone growth disease or disorder, but may be susceptible to suffering from a bone growth disease or disorder. For example, a subject susceptible to suffering from a bone growth disease or disorder can be a subject that has been diagnosed as having an increased risk of developing a bone growth disease or disorder. A determination of whether a given subject is suffering from a given bone growth disease or is susceptible to a given bone growth disease can be made by those of skilled in the art based on clinical symptoms and/or other standard diagnostic tests which may vary depending on the particular disease in question.

As used herein, “bone growth disease” or “bone growth disorder” refers to a disease or condition associated with abnormality of the bone that can be treated by increasing bone mass and/or bone growth. A wide variety of bone growth disease and disorders are known to those skilled in the art. Examples of bone growth diseases and disorders include, but are not limited to: Achondrogenesis; Achondroplasia; Acrodysostosis; Acromesomelic Dysplasia (Acromesomelic Dysplasia Maroteaux Type, AMDM); Atelosteogenesis; Campomelic Dysplasia; Cartilage Hair Hypoplasia (CHH) (Metaphyseal Chondrodysplasia, McKusick type); Chondrodysplasia Punctata; Cleidocranial Dysostosis; Conradi-Hunermann Syndrome; Cornelia de Lange; Cranioectodermal dysplasia; Desbuquois syndrome; Diastrophic Dysplasia; Dyggve-Melchior-Clausen; Dyssegmental Dysplasia; Ellis van Creveld Syndrome (Chondroectodermal Dysplasia, EVC); Growth Hormone Deficiency; Hallerman-Streiff Syndrome; Hunter Syndrome (MPS II); Hurler-Scheie Syndrome (MPS I); Hypochondrogenesis; Hypochondroplasia; Hypophosphatasia; Hypophosphatemia; Hypopituitary; Hypothyroidism; Jarcho-Levin Syndrome (Spondylothoracic Dysplasia, Spondylocostal); Jeune Syndrome (Asphyxiating Thoracic Dysplasia; Asphyxiating Thoracic Dystrophy); Kniest Dysplasia; Laron Dwarfism; Larsen Syndrome; Leri-Weill Dyschondrosteosis (Mesomelic Dwarfism, Madelung Deformity); Lethal Skeletal Dysplasias; Maroteaux-Lamy (MPS VI) (MPS VI); Mesomelic Dysplasia; Metaphyseal Chondrodysplasia-Jansen Type; Metaphyseal Dysplasia-Schmid Type; Metatropic Dysplasia; Morquio Syndrome (MPS IV); Mucopolysaccharidoses; Multiple Epiphyseal Dysplasia (MED); Osteogenesis Imperfecta (OI); Pituitary Dwarfism; Precocious Puberty; Primordial Dwarfism (Microcephalic Osteodysplastic Primordial Dwarfism, MOPD); Pseudoachondroplasia; Rhizomelic Chondrodysplasia Punctata; Rickets; Robinow dwarfism/syndrome; Russell-Silver Syndrome; SADDAN: Severe Achondroplasia with Acanthosis Nigricans and Developmental Delay; Schmike Immuneosseous Dysplasia; Seckel Syndrome; Short Rib Polydactyly; Shwachman-Diamond Syndrome; Spondyloepimetaphyseal Dysplasia-Strudwick (SEMD); Spondyloepimetaphyseal dysplasias; Spondyloepiphyseal Dysplasia; Spondyloepiphyseal Dysplasia Congenita (SED-Congenita, SEDC); Spondyloepiphyseal Dysplasia Tarda (SED-Tarda, SEDT, SED-L); Spondylometaphyseal Dysplasia-Corner Fracture Type (SMD, SMD-Corner Fracture Type, SMD-Sutcliffe Type); Spondylometaphyseal Dysplasia-Kozlowski (SMD-Kozlowski, SMDK); Thanatophoric Dysplasia; Trichorhinophalangeal Syndrome (Langer-Giedion syndrome); Turner Syndrome; Type II Collagenopathies.

In some embodiments, the subject has been diagnosed as having bone growth disease or disorder selected from the group consisting of osteogenesis imperfecta, disorders caused by increased osteoclastogenesis or bone loss associated with inflammatory conditions, infection, genetic and age-related bone disorders such as osteoporosis, osteopenia, Paget's disease, metastatic bone cancer, myeloma bone disease, bone fracture healing, and bone graft repair.

Bone disease or disorders characterized by a loss of bone mass also encompass abnormalities in the strength and structures of the bones. Examples include, but are not limited to, decreased bone mass, change in bone density, bone softness, tumors on bones and abnormal bone architecture. Additionally or alternatively, a diagnosis of a given bone disease can be made by a physician, nurse, or veterinarian, depending on the subject under consideration.

The level of or changes in bone mass can be useful to determine if a subject is suffering from a bone growth disease or disorder, or to evaluate treatment of a bone growth disease or disorder by decreasing bone resorption. Bone mass may be measured in mammalian subjects (e.g. humans) using standard techniques (e.g. dual energy X-ray absorptiometry (DXA)). In particular, DXA may be used for diagnosis, prognosis (e.g. fracture prediction), monitoring the progression of a bone disease, and/or assessing responses to treatment. Categorization of subjects into diseased and non-diseased states based on bone mass (i.e. bone material density) can be made on the basis of standard classification systems including those published by the World Health Organization (see, for example, World Health Organization Technical Report Series 921(2003), Prevention and Management of Osteoporosis). For example, a diagnosis of osteoporosis may be based on BMD that is two standard deviations or more below a young adult reference mean.

The composition used to inhibit bone resorption can include any of the compositions described herein. In some embodiments, the gallium compound is selected from the group consisting of gallium acetylacetonate, gallium nitrate, and gallium citrate. In further embodiments, the gallium compound of the composition is gallium acetylacetonate. In additional embodiments, the cellulose-based hydrogel of the composition is a methylcellulose hydrogel. In yet further embodiments, the cellulose-based hydrogel comprises a bone-seeking ligand.

In some embodiments, the method of inhibiting bone resorption also includes administration of one or more additional bone growth stimulating agents. The term “bone growth-stimulating agent” is intended to include any material that stimulates and encourages the development and functional maintenance of bone, and mature osteoclasts in bone. Examples of bone growth-stimulating agents include, without limitation: growth factors; cytokines and the like, such as members of the Transforming Growth Factor-beta (TGF-β) protein superfamily including any of the Bone Morphogenic Proteins (BMP) and members of Glycosylphosphatidylinositol-anchored (GPI-anchored) signaling proteins including members of the Repulsive Guidance Molecule (RGM) protein family; other growth regulatory proteins, and bisphosphonates.

Because patients with a bone disease or disorder have heterogeneous clinical manifestations and varying clinical outcomes, the treatment given to a subject may vary, depending on their prognosis. The skilled clinician will be able to readily determine without undue experimentation specific secondary agents, types of therapy that can be effectively used to treat an individual subject with a bone disease or disorder.

The method for inhibiting bone resorption includes in vivo placement of a composition for inhibiting bone resorption, or an implantable orthopedic device coated with a composition for inhibiting bone resorption, as described herein for bioengineering, restoring or regenerating bone. In particular aspects of the method, bioengineering, restoring or regenerating bone is in vitro or ex vivo, including placement under body fluid conditions. The method includes positioning the composition for inhibiting bone resorption, or an implantable orthopedic device coated with a composition for inhibiting bone resorption to provide structural support for nearby tissue. In some embodiments of the method, the compositions are used for dental and orthopedic implants, craniomaxillofacial applications and spinal grafting, and said composition is suitable to promote bone regeneration and repair.

Preferably the method of inhibiting bone resorption occurs under aseptic conditions. “Aseptic” as the term is used herein, refers to methods to control or reduce the microbial bioburden in an environment. Tissues processed “aseptically” are tissues processed using sterile instruments, and special environmental surroundings (including for example “clean room technologies”).

In some embodiments, the composition is in an injectable form, and the step of contacting a site comprises administering the composition by injection to the site in need of inhibition of bone resorption. “Injectable” refers to the ability of certain compositions for inhibiting bone resorption of the present invention to be introduced at an implant site under pressure (as by introduction using a syringe). An injectable composition of the present invention may, for example, be introduced between elements or into a confined space in vivo (i.e., between pieces of bone or into the interface between a prosthetic device and bone, among others). For example, the compositions may be injected into the vertebral body for prevention or treatment of spinal fractures, injected into long bone or flat bone fractures to augment the fracture repair or to stabilize the fractured fragments, or injected into intact osteoporotic bones to improve bone strength. The injectable composition can be extruded through a syringe and/or a syringe having at least a 13 gauge tube/needle coupled thereto.

In further embodiments, the site in need of decreased bone resorption is a dental site. Decreasing bone at a dental site can improve the repair of teeth, or bone tissue near the teeth. The bone resorption inhibiting composition can be used as part of a bone repair process following extraction of a tooth and/or placement of a dental prosthesis, or for repairing dental bone defects such as bone loss from moderate or severe periodontitis.

### Scaffolds

In some embodiments, the composition for inhibiting bone resorption is configured as a tissue scaffold. A tissue scaffold is a support structure that provides a matrix for cells to guide the process of bone tissue formation in vivo. The morphology of the scaffold guides cell migration and cells are able to migrate into or over the scaffold, respectively. The cells then are able to proliferate and synthesize new tissue and form bone and/or cartilage. While there are many criteria for an ideal tissue scaffold for bone tissue repair, an important characteristic is the presence of a highly interconnected porous network with both pore sizes and pore interconnections large enough for cell migration, fluid exchange, and eventual tissue in-growth and vascularization.

The composition for inhibiting bone resorption can be molded or otherwise shaped during preparation to have any desired configuration as a tissue scaffold. Typically, the material is molded to have the shape of the bone or bone-like material that it is being substituted for. However, the scaffold material can also be used for cosmetic work or “bioengineering,” where a support structure is provided for the creation of new tissue rather than the replacement or regeneration of existing tissue. In some embodiments, the tissue scaffold may be seeded with harvested bone cells and/or bone tissue, such as for example, cortical bone, autogenous bone, allogenic bones and/or xenogenic bone. For further information regarding suitable tissue scaffolds for bone repair or regeneration, see for example U.S. patent applications Ser. Nos. 11/793,625, 12/193,794, 13/908,627, or 14/216,451, the disclosures of which are incorporated herein by reference.

In some embodiments, the scaffold is bioresorbable. Bioresorbable, as used herein, refers to the ability of the scaffolds to be gradually degraded by physiological processes in vivo, to allow the replacement of the biocompatible material with native tissue. For example, if the scaffold is used to replace bone, the scaffold may be gradually degraded while osteoblasts rebuild bone tissue in its place (i.e., bone remodeling).

Examples have been included to more clearly describe a particular embodiment of the invention and its associated cost and operational advantages. However, there are a wide variety of other embodiments within the scope of the present invention, which should not be limited to the particular examples provided herein.

### EXAMPLES

### Example 1

### Efficacy Assessment of Methylcellulose-Based Thermoresponsive Hydrogels Loaded With Gallium Acetylacetonate in Osteoclastic Bone Resorption

The inventors set out to assess hydrogels as delivery systems for GaAcAc. Unique characteristics of hydrogels can be attributed to the type of materials used in preparation and method of synthesis as well as the ability to respond to changes in the environment like temperature, pH, or presence of enzyme and specific biomarkers. Zhang et al., Gels, 7(3): p. 77(2021 ). The inventors paid particular attention to thermoresponsive hydrogels that respond to changes in temperature by converting from solution at room temperature to gel at body temperature. Liow et al., ACS Biomaterials Science & Engineering, 2(3): p. 295-316 (2016). The attractiveness of thermoresponsive hydrogels is that they can be easily injected locally into the desired bone site, at room temperature in the form of a solution and transition to gel at the bone site with temperature change, thereby localizing the therapeutic agent at the desired bone site The biocompatibility and affordability of cellulose-based hydrogels make them a great choice for our study. In general, cellulose-based hydrogels are formed by crosslinking aqueous solutions of cellulose ethers such as methylcellulose and hydroxypropyl methylcellulose. Sannino et al., Materials, 2(2): p. 353-373 (2009). Cellulose-derived hydrogels form a mesh-like network that traps the drug moieties within its structure until the surrounding environment enables its release in a controlled manner. Thirumala et al., Cells, 2(3): p. 460-475 (2013). Thus, we fabricated methylcellulose-based hydrogels that were evaluated as delivery systems for GaAcAc based on (i) biocompatibility towards pre-OB and pre-OC cells, (ii) effects of GaAcAc loading on the hydrogel's thermoresponsiveness and (iii) efficacy assessment in inhibiting OC differentiation and function. Overall, we applied in-vitro and in-vivo approaches to assess the suitability of methylcellulose-based hydrogels as a delivery for GaAcAc in osteoclastic bone resorption.

### Experimental

### Materials

RAW 264.7 (murine macrophage-osteoclast precursor) cells were procured from ATCC; Manassas, VA. Gallium acetylacetonate (GaAcAc), Methylcellulose powder, penicillin-streptomycin, 3-(4,5-Dimethylthiazol-2yl)-2,5-diphenyltetrazolium bromide (MTT), Fast Red Violet Salt, toluidine blue and naphthol AX-MX, protease inhibitor cocktail, neutral red dye, 2-mercaptoethanol were all obtained from Sigma-Aldrich (St. Louis, MO). Dimethyl sulfoxide (DMSO), phosphate-buffered saline (PBS), bovine serum albumin (protease free), alpha-modified eagle's medium (a-MEM), Dulbecco's Modified Eagle's Medium (DMEM), and p-nitrophenyl phosphate were all purchased from GIBCO & Fisher Scientific (Pittsburgh, PA). RANKL & M-CSF was bought from R&D Technologies (Minneapolis, MN, USA), and fetal bovine serum (FBS) was obtained from Atlanta Biologicals (Lawrenceville, GA). Bovine cortical bone slices were purchased from BioVendor R&D® (Asheville, NC). NFAT2, TRAF6, cFos & IgG-HRP, GAPDH antibodies were procured from Cell Signaling Technology (Danvers, MA), TRAP antibody from Abcam, (Cambridge, United Kingdom). Indocyanine green (ICG) was purchased from Cayman Chemical Company (Ann Arbor, MI). RIPA lysis/extraction buffer was purchased from ThermoFisher Scientific (Waltham, MA), 24 well transwell plates were procured from Corning Inc (Corning, NY), Immobilon ® Forte Western HRP substrate was procured from Millipore (Burlington, MA). 4× laemmli buffer, TransBlot ® Turbo™ Mini-size PVDF membrane, Trans-Blot Turbo Mini-size Transfer Stacks, and 10× Transfer buffer were procured from Bio-Rad (Hercules, CA). Immunoblotto non-fat dry milk was procured from Santa Cruz Biotechnology (Dallas, TX), and Immobilon® Forte Western HRP substrate was procured from EMD Millipore (Burlington, MA). BCA Pierce Assay kit was procured from Thermo Scientific Pierce™, Rockford, IL. Nitric acid (65% v/v) was procured from Fluka Analytics (Buchs, CH), Yttrium and ICP grade water were procured from Inorganic Ventures (Christiansburg, VA). Mouse CTSK/Cathepsin K (Sandwich ELISA) ELISA Kit was procured from Lifespan Biosciences (Seattle, WA).

### Preparation of Methylcellulose Hydrogel (mh) and Gaacac-loaded Hydrogels (GaMH)

Methylcellulose hydrogels (MH) were prepared via the dispersion technique by adapting earlier reported methods. Contessi et al., Materials Letters, 207: p. 157-160 (2017). Briefly, the methylcellulose powder was weighed and dispersed in preheated (55° C.) PBS (2-12 % w/v), followed by overnight spinning (700 RPM) at 4° C. The resultant gels were maintained in 4° C. before further testing. The same procedure was followed to prepare GaAcAc-loaded hydrogels (GaMH) with a slight modification of dropwise addition of various concentrations of GaAcAc into the hydrogels under stirring.

### Rheological Measurements

Rheological characterization of different concentrations of MH and GaMH with different concentrations of GaAcAc were analyzed using a Haake Mars Rheometer (222-1912, Thermo Fisher Scientific). The following parameters were conducted; dynamic shear oscillation and temperature-dependent analysis of modules. The measurements were carried out using minimal strain to characterize the viscoelastic properties using serrated plates of 40 mm diameter with a plate-to-plate distance of 0.9 mm. Temperature-dependent studies of the storage (G′) and loss (G″) modulus were conducted using oscillatory shear with temperatures ranging from 20 to 50° C. (heating rate: 2°/min) at a constant frequency (1 Hz) and shear strain (1 mrad). G′ is the measure of elasticity of the material and its ability to store energy, whereas G″ is the ability is to release energy. By measuring the G′ and G″ moduli over a temperature range, the sol-gel transition of the hydrogel can be assessed. Stojkov et al., Gels, 7(4): p. 255(2021 ) The point at which G′ is equal to G″ is defined as gelation temperature where it indicates the phase transition of hydrogel due to its response to the change in temperature.

The data was analyzed and presented with the help of Rheology Data Manager software. Based on the data obtained, the percentage of methylcellulose hydrogel and drug-loaded hydrogel were selected for further analysis. Kim, M. H., et al., International journal of biological macromolecules, 109: p. 57-64 (2018); Park et al., Carbohydrate polymers, 157: p. 775-783 (2017); Yeo, Y. H. and W. H. Park, Carbohydrate Polymers, 258: p. 117705(2021 ).

### Gelation Test of Hydrogel

The test-tube tilting method was applied to observe the ability of the hydrogels to undergo reversible solution-to-gel transition at different temperature conditions. Starting with 4° C., the gel was sealed in a glass vial. The phase of the gel was observed by tilting the vial several times. The process was repeated at 25° C. and 37° C.

### In-vitro Degradation of MH Hydrogel

To characterize the in vitro degradation of MH, we maintained the hydrogels at 37° C. and measured the initial weight of the solidified gel. Subsequently, we added 1 mL of PBS (pre-warmed at 37° C.) on top of the solidified gel and maintained it at 37° C. over time. At pre-determined time intervals, we aspirated the PBS layer on top of the solidified gel before measuring the weight of the hydrogel layer over time. The extent of MH hydrogel degradation was calculated using the initial weight of the hydrogel layer and the weight measured at different time intervals during the experiment.

### In-vitro Gaacac Release From Hydrogel

The rate and extent of GaAcAc release from GaMH were assessed. Briefly, we maintained GaMH at 37° C. Subsequently, we added 1 mL of pre-warmed (37° C.) PBS on top of the solidified gel and subjected it to gentle agitation of 50 rpm in an incubator maintained at 37° C. The PBS layer was aspirated at different time intervals and replenished with fresh pre-warmed PBS. The amount of GaAcAc released at various time points and were analyzed for GaAcAc released using a UV-Vis spectrophotometer at 278 nm.

### Cell Culture

RAW 264.7 (pre-osteoclast) and MC3T3 (pre-osteoblast) cells were cultured and maintained in DMEM & AMEM supplemented with 10% FBS and 1% PenStrep in a humidified incubator at 37° C. and 5% CO₂ conditions. RAW and MC 3 T3 cells, upon 70-80% confluency, were gently detached using a cell scraper and/or 0.25% v/v trypsin/EDTA. The detached cell suspension was collected and centrifuged at 1500 RPM for 5 minutes and seeded accordingly to the requirement for each experiment.

### Biocompatibility Assessment

We assessed the biocompatibility of GaAcAc in pre-osteoclastic cells (RAW 264.7) and pre-osteoblastic cells (MC3T3). Cells were incubated (37° C.) with various concentrations of GaAcAc (10-50 μg/mL). After the stipulated time of incubation, cell viability was measured by MTT assay using untreated control cells as a reference.

The biocompatibility of MH was carried out in pre-OC cells (RAW 264.7) by either incubating MH leachates with cells or introducing MH to cells via transwell inserts. To collect MH leachates, 1 mL serum-free media was added on top of solidified hydrogels and incubated (37° C.) for 24 hrs. The supernatant was collected and filtered. RAW 364.7 cells were plated and incubated overnight in a complete growth medium. Subsequently, the content of each well was aspirated and treated with MH-filtered leachate supplemented with FBS and incubated for a predetermined time. The percentage of cell viability was assessed using untreated control cells as a reference.

To add MH to cells via transwell inserts, cells were seeded overnight at a density of 5000 cells/well and incubated overnight. The next day, 100 μL of MH was added to the top compartment of the set and incubated for gelation before assembling the top and bottom compartment of the transwell plate. The assembly was then incubated for 24, 48, and 72 hrs. After each time point, the contents of each well were aspirated, and the cell viability assessment was carried out via MTT. The percentage of cell viability was calculated based on control (untreated) cells as a reference (100% viability).

### OC Differentiation Studies

### Raw 264.7 Cells Differentiated OCs

RAW 264.7 (Pre-OC) cells were seeded at a density of 1.5×10⁵ cells/cm² (day 0) in a 24-well bottom dish of an insert plate and incubated overnight for adherence. Subsequently, (day 1), cells were treated with growth media supplemented with 30 ng/ml of RANKL (positive control) alone or RANKL (30 ng/ml) together with various treatments-GaAcAc solution, MH, or GaMH. Cells that did not receive RANKL supplementation served as negative controls. After the initial treatment and RANKL supplementation, cells in all treatment groups (except untreated control) received RANKL supplementation on day 3 to induce OC formation. On day 4, mature OCs were fixed at 4% paraformaldehyde for 10 minutes at room temperature and evaluated for the extent of OC differentiation based on tartrate-resistant acid phosphatase (TRAP) activity and stained with TRAP staining solution. TRAP (pink colored) stained and multinucleated (≥3 nuclei) cells according to earlier reported methods. Abdelmagid et al., J Biol Chem, 290(33): p. 20128-46 (2015).

### Hematopoietic Stem Cells Differentiated OCs

All mice were housed and maintained at Northeast Ohio Medical University according to the guidelines of the Institutional Animal Care and Use Committee. Bone marrow cells were flushed from the femora, and tibiae were extracted from euthanized C57BL/6 mice (male, 6-8 weeks) as described previously. Xing L. and B.F. Boyce, Methods Mol Biol, 1130: p. 307-313(2014). The flushed cells were incubated overnight to separate hematopoietic (in supernatant) and mesenchymal (adherent) stem cells. The supernatant was then collected and seeded at the required density based on the type of well plate used (96 well plate: 2×10⁵ cells/well & 24 well plate: 1.5×10⁶ cells per well-day 0) in culture media supplemented with 30 ng/ml of M-CSF. On day-4, the contents of wells were aspirated and treated with culture media supplemented with the following: 30 ng/ml M-CSF, 30 ng/ml RANKL alone (control) or in addition with either GaAcAc solution, MH or GaMH (Day 4). On day-6, fresh media supplemented with M-CSF & RANKL were added to the differentiating cells. On day-7, the plates were fixed and evaluated for TRAP activity and counted for the number of TRAP-stained multinucleated osteoclasts formed similarly as mentioned above.

### Mechanistic Assessment of OC Differentiation

To investigate the effects on OC differentiation, Pre-OC (RAW 264.7) cells were seeded, treated, and differentiated with various treatments as described under OC differentiation. Afterward, cells were lysed with 100 μL of RIPA buffer containing protease inhibitor. The lysates were collected and centrifuged at 1500 RPM for 10 minutes and analyzed for their protein content via the BCA protein assay kit. Protein samples (20 μg) were separated via 10% SDS-page gel and transferred onto a polyvinylidene fluoride (PVDF) membrane. The membrane was blocked with a respective blocking buffer, either 5% BSA or non-fat dry milk, for an hour. After blocking, the membranes were washed with 1× TBST buffer and later probed with primary antibodies; NFAT2, TRAP, TRAF6 and cFos with GAPDH as a loading control, diluted with 2% respective blocking buffer overnight at 4° C., followed by a 1-hour incubation with secondary HRP conjugated anti-rabbit IgG secondary antibody. The blots were visualized using the Fluorchem system [63].

### Pit Resorption Assay for OC Function

To assess the effects of various treatments on OC function, we assessed the possible anti-resorptive effect via pit resorption assay. Briefly, bovine cortical bone slices were cleaned and sterilized as per the manufacturer's instructions and placed in a 24-well plate. The study was carried out using OC derived from either RAW 264.7 differentiation or murine hematopoietic stem cells (mHSCs).

Using OCs from RAW 264.7 cells, the detached pre-OC cells were seeded on top of the sanitized bovine cortical bone slice using 1.5×10⁵ cells/cm² supplemented with a complete growth medium on day 0. On the next day (day 1), the media was aspirated from cells followed by treatment with fresh media supplemented with RANKL alone or RANKL with one of the treatments: GaAcAc, MH or GaMH. Subsequent treatment with complete media containing RANKL occurred every two days for seven days. After seven days, the wells were aspirated and processed for further analysis.

Separate studies with mHSCs involved seeding mHSCs on top of bone slices at the required amount based on the plate used (96 well plate: 2×10⁵ cells/well; 24 well plate: 1.5×10⁶ cells/well) in complete AMEM media supplemented with 30 ng/mL M-CSF (Day 0). After 3 days, the wells were treated with media supplemented with RANKL & M-CSF with or without GaAcAc alone, MH or GaMH on day 4. The wells were aspirated on day 6 and replenished with media supplemented with RANKL & M-CSF. The next day, the wells were aspirated and replenished with media containing RANKL; this was repeated for the next two days. On day 10, the wells were aspirated and the bone chips were fixed and stained.

To assess the osteoclastic resorptive effect of the wells with or without treatment, the bone slices were fixed with 10% formaldehyde and initially TRAP-stained, as reported. The bone slices were thoroughly cleaned with cotton swab and strained with 0.5% toluidine blue stain and incubated for three minutes at room temperature. The slices were then washed with water five times and patted dry. The pits formed on the bone slices, depicting OC function, were evaluated.

### Mechanistic Assessment of OC Function

Cathepsin K (CTSK) assay was conducted during bone resorption studies (as described above) as a useful tool to assess the effects of GaMH treatment on OC function. Supernatant from treated/untreated wells after the third treatment of differentiation factors was collected and spun down at 2000g for 10 minutes at 4° C. The supernatant was then assessed for its CTSK concentration using the mouse CTSK/Cathepsin K Sandwich ELISA kit as per manufacture protocol.

### In-vivo Retention GaMH

In-vivo retention studies were carried out in healthy BALB/c mice using indocyanine green (ICG) as the marker with monitoring via the in-vivo imaging system (IVIS) Lumina XRMS series III (PerkinElmer, USA). All animal studies were performed according to a protocol approved by the Institutional Animal Care and Use Committee of Northeast Ohio Medical University. Animals were briefly anesthetized with 2% inhalational isoflurane and administered with 100 μL of ICG solution (20 μg/mL in PBS) as a solution or loaded in GaAcAc loaded methylcellulose hydrogel (ICG-GaMH).

All treatments were administered by a single periosseous (intramuscular, bone adjacent) injection in the lower right hindlimb. The animals were observed via IVIS at a selected time point to obtain total radiant efficiency of the injection site with an excitation set at 780 nm and emission at 845 nm based on the earlier reported method. Okubo et al., International Journal of Pharmaceutics, 575: p. 118845(2020 ).

### In-Vivo GaAcAc Biodistribution Analysis

BALB/c mice were randomly distributed into groups that received 100 μL volume of either GaAcAc solution (100 μg/mL in PBS) or GaMH (loaded with 100 μg/mL GaAcAc). All treatments were administered by a single, periosseous (intramuscular, bone adjacent) injection in the lower right hindlimb. At different time intervals post injection (2 hrs and 24 hrs), mice were sacrificed. From each mouse, we harvested the injection site (lower right hindlimb) as well as a non-injection site (lower left hindlimb). The concentrations of Ga from injection and non-injection sites were analyzed by inductively coupled plasma atomic emission spectroscopy (ICP), (Thermo Scientific iCAP 7400) and normalized by weight of tissue sample. The process of acid digestion of samples was developed using our earlier reported method. Wehrung et al., Journal of biomedical nanotechnology, 9(6): p. 1029-1040 (2013). In developing the ICP analysis process we used 0.83 ppm of yttrium as an internal standard.

### Data Analysis

Statistical analysis was carried out using Student's t-test or a single factor ANOVA. Student-Newman-Keuls posthoc test was applied for multiple comparisons while assessing significance at a p-value of <0.05. The graphs were prepared with GraphPad Prism 6 (GraphPad Software, CA).

### Results

### In-vitro Characterization of GaAcAc in Pre-osteoclastic and Pre-osteoblastic Cells

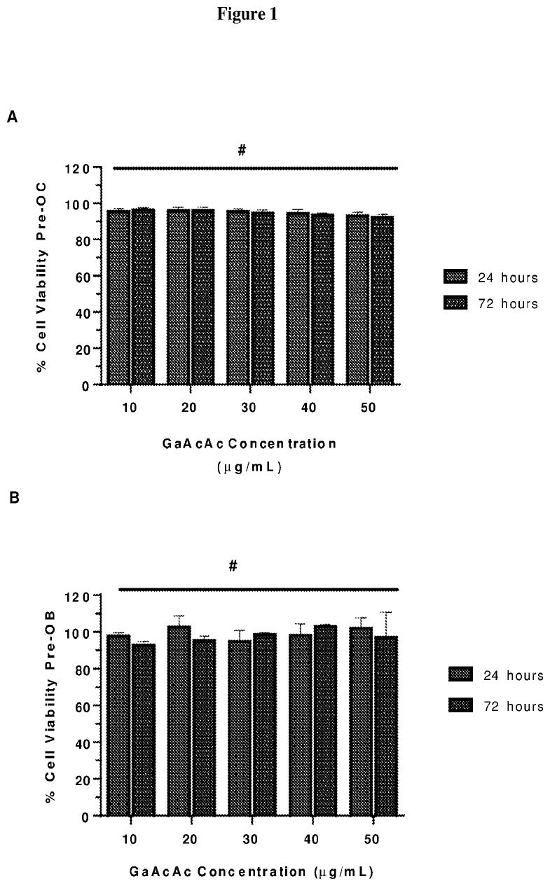

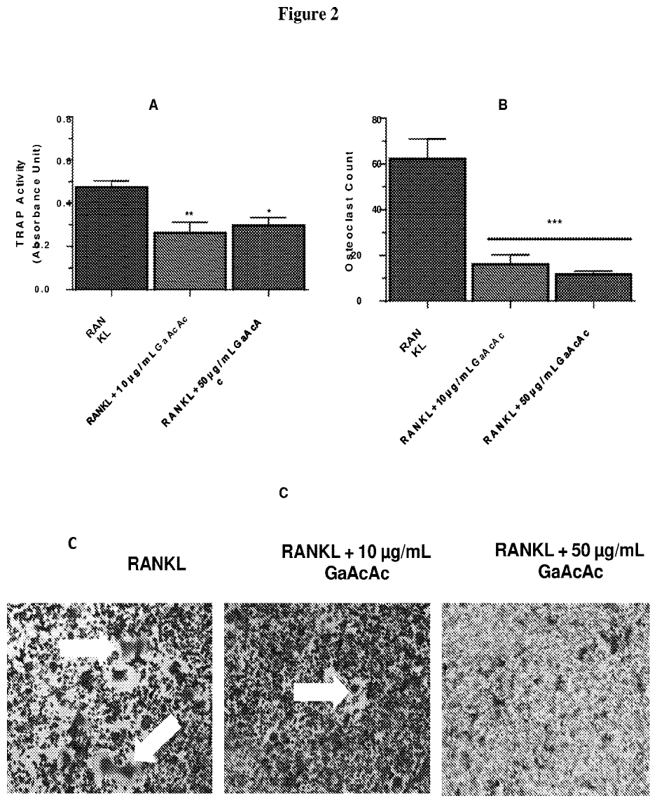

We investigated the effects of various concentrations of GaAcAc on the viability of pre-osteoclastic (RAW 264.7) or pre-osteoblastic (MC3T3) cells (FIG. 1). GaAcAc concentrations ranging from 10-50 μg/mL (37° C.) did not affect the viability of pre-OC (FIG. 1A) or pre-OB (FIG. 1B) cells. Using this GaAcAc concentration window, we explored the effect on OC differentiation as monitored by a notable marker of OC differentiation, tartrate-resistant acid phosphate (TRAP). The differentiation of RAW 264.7 cells into OCs occurred by treatment with RANKL treatment which was supplemented until the differentiation was complete. The differentiated cells were assessed for TRAP activity spectrophotometrically. The trend was substantiated by the number of mature OCs (OC count). The OC count was based on the identification and number of TRAP-stained multinuclear cells (pink color and nuclei≥3) compared between treatment groups to positive control (FIG. 2). GaAcAc treatments were introduced (once) during differentiation with RANKL supplementation. Our data indicate that GaAcAc treatment (at 10 μg/mL and 50 μg/mL) exhibited a remarkable decrease in TRAP activity compared to positive control cells that received RANKL supplementation alone (FIG. 2A; p>0.001).

Further assessment of the extent of OC differentiation was based on the number of TRAP-stained OCs (FIG. 2B). Treatment with 10 μg/mL or 50 μg/mL GaAcAc solution resulted in a significant reduction in the number of OCs after differentiation (p<0.0001, FIG. 2B) when compared to positive control cells that were differentiated without GaAcAc treatment. This inhibitory effect of GaAcAc on OC differentiation was observed in the representative images after differentiation (FIG. 2C) which showed the presence of TRAP-stained multinuclear cells in positive control wells (RANKL) and a decrease in TRAP-stained cells with 10 μg/mL GaAcAc treatment (FIG. 2C). We observed a complete absence of multinucleated and TRAP-stained cells in wells that received the higher GaAcAc concentration (50 μg/mL, FIG. 2C). The data obtained from OC differentiation with mHSCs followed the same trend indicating that GaAcAc treatment suppressed OC differentiation and OC counts. The effect of GaAcAc on OC differentiation using mHSCs was pronounced at 50 μg/mL GaAcAc than at 10 g/mL. Additional results showed that suppression of OC differentiation by GaAcAc and OC count was dose-dependent, with a significant inhibition (p<0.0001) observed at GaAcAc concentrations that were greater than 25 μg/mL.

### Preparation and Characterization of Methylcellulose-Based Hydrogel

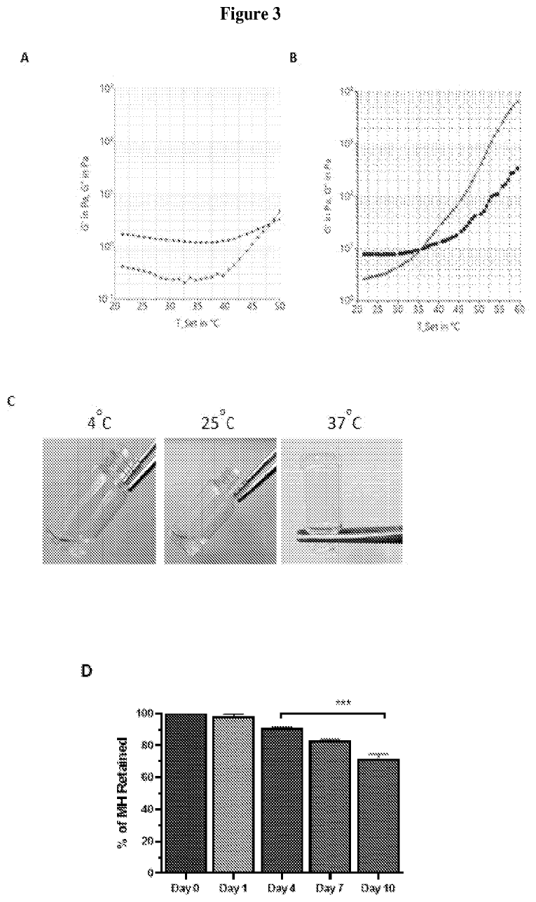

Different concentrations of methylcellulose (MC), ranging from 2% to 12 % w/v, were used to prepare hydrogels. At MC concentrations between 2% w/v and 8% w/v, we prepared suitable hydrogels. MC concentrations above 8% w/v did not result in acceptable hydrogel formulations due to high viscosity that would not be useful practically due to the injectability consideration. All the hydrogels were characterized by thermoreversibility using rheological assessment of storage (G′) and loss (G″) moduli parameters acquired as temperature increased from 25° C. to 60° C. while noting the temperature at which the moduli will crossover (FIG. 3). At MC concentrations lower than 6% w/v, there was no moduli crossover as temperature increased. Hydrogels prepared at MC concentrations 6% w/v exhibited moduli crossover (the gelation temperature) at temperatures around 50° C. (FIG. 3A). Meanwhile, the gelation temperature for hydrogels prepared at MC concentration of 8% w/v was 37° C.

Further characterization of the thermoreversibility of the lead hydrogel formulation (MH prepared with 8% w/v MC) was carried out using the vial inversion method. The hydrogel was incubated at three different temperatures: 4° C., 25° C., and 37° C. At the same time, we assessed the fluidity by tilting the vial and taking pictures (FIG. 3C). The transition from the solution phase to a solid phase (gel) occurred as the temperature increased as captured for blank hydrogel (MH) (FIG. 3C). In-vitro degradation of blank hydrogel (MH) was evaluated with a PBS top layer (37° C.) followed by subsequent measurement of the extent to which the MH hydrogel layer will diminish over the course of ten days. We observed that the weight of the hydrogel layer decreased consistently over time. The weight on day 0 was set as a reference (100%, FIG. 3D) remained intact (p>0.05) till the following day (day 1). The average percent hydrogel layer retained on day 4 was 90.2% (FIG. 3D) which gradually decreased to an average of 70% on day 10, indicating a loss of 15% hydrogel weight by day 10 (FIG. 3D). The biocompatibility of the hydrogel (MH) was demonstrated based on the viability of pre-OCs (RAW 264.7) and pre-OB (MC3T3) cells using MTT or neutral red dye uptake methods.

### Preparation and Characterization of Gaacac-loaded Hydrogels (gamh)

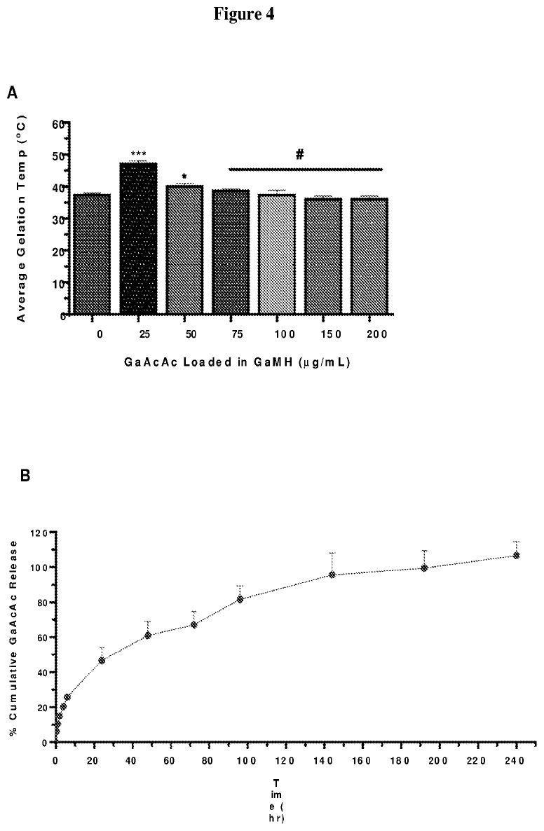

Hydrogels loaded with different concentrations of GaAcAc (GaMH), ranging from 25 μg/mL to 200 μg/mL, were prepared and characterized. Rheological parameters, storage (G′), and loss (G″) moduli, with increased temperature (20° C. to 60° C.) were obtained. From the moduli readings, we obtained the average gelation temperature of GaMH at different concentrations of GaAcAc. At GaAcAc concentrations (25-50 μg/mL), the average gelation temperatures increased slightly, compared to blank hydrogels (p<0.05) and later remained comparable to blank hydrogels. As we increased GaAcAc concentrations from 75 μg/mL to 200 μg/mL, the average gelation temperatures were comparable to blank hydrogels (p>0.05; FIG. 4A). GaMH formulation loaded with a GaAcAc concentration of 100 μg/mL was selected as the lead for subsequent studies. Further characterization of the thermoreversibility of the selected GaMH (containing GaAcAc 100 μg/mL) was carried out using the vial inversion method with incubation at three different temperatures. The images showed fluidity of GaMH at 4° C. and 25° C., consistent with the solution phase, but not at 37° C., consistent with the gel or solid phase. The rate of GaAcAc release from GaMH was evaluated by maintaining the hydrogel formulations at the gel phase (37° C.) throughout the study with gentle shaking at 50 RPM with a PBS layer on top. Within 6 hrs, we observed an average of 25% GaAcAc release which gradually increased to an average of 46% GaAcAc release by 24 hrs. The rate of GaAcAc release slowed down afterward resulting in an average 61, 67 and 81% at 48, 72 and 96 hrs, respectively (FIG. 4B).

### Efficacy Assessment in OC Differentiation and Function

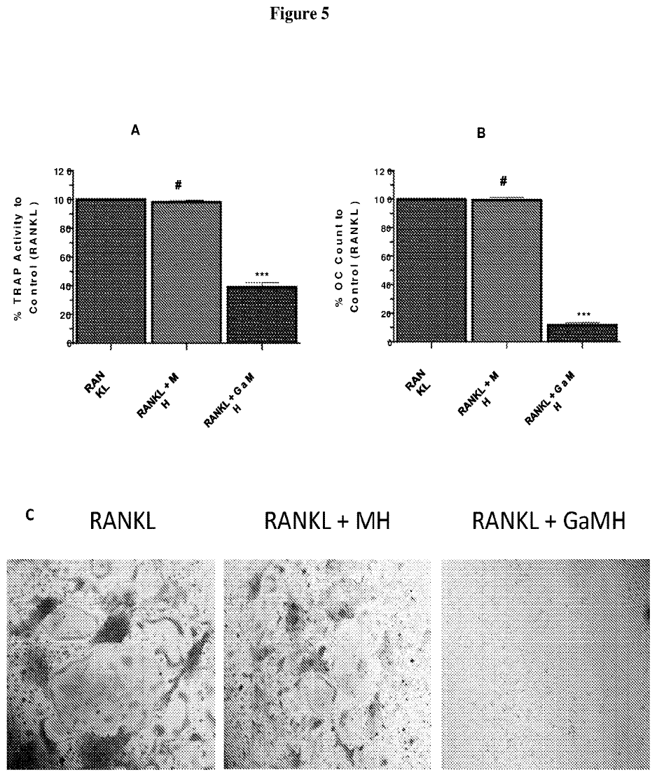

The initial observation showed that blank hydrogels (MH) when added during differentiation of mHSCs to OCs did not interfere with the differentiation process (FIG. 5). TRAP activity and OC counts following M-CSF and RANKL supplementation with MH treatment were comparable to the observation with the positive control cells that were differentiated without MH treatment (FIGS. 5A and 5B). A similar trend was observed with differentiation of RAW 264.7 cells to OCs with or without MH treatment. However, with GaMH treatment, there was a significant decrease in the TRAP activity and OC counts compared to positive control cells (p<0.0001, FIGS. 5A and 5B). Further, the presence of TRAP-stained multinuclear cells was unaffected in wells treated with MH; however, the wells treated with GaMH significantly decreased the presence of any multinucleated cells (FIG. 5C).

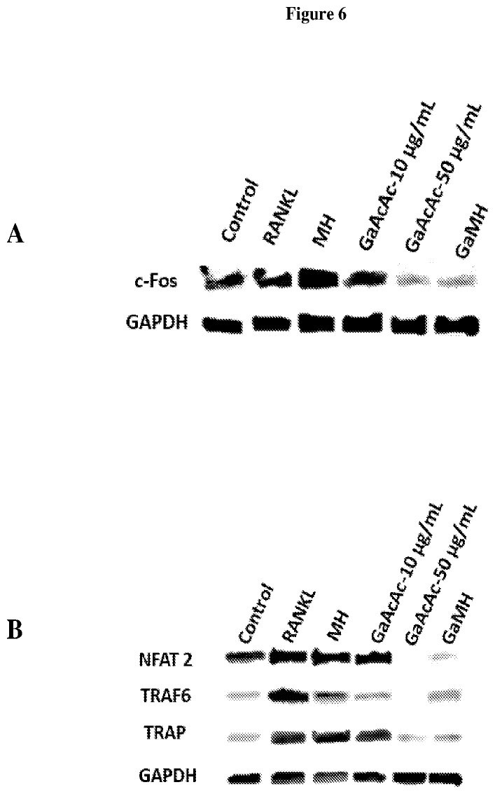

To analyze the mechanistic basis of the observed trends in OC differentiation and function, we performed western blotting analyses focusing on key markers involved in OC differentiation such as TRAP, NFAT2 and cFos (FIG. 6). We observed that treatment with blank hydrogel (MH) during differentiation did not affect the expression of any of these markers compared to the positive control cells (with RANKL supplementation alone). Treatment with 10 μg/mL GaAcAc solution during differentiation resulted in a slight decrease in expression levels of the markers when compared to positive control. Meanwhile, treatment with 50 μg/mL GaAcAc or GaMH (loaded with 10 μg/mL GaAcAc) resulted in downregulation of expression levels of these markers when compared to a positive control (FIG. 6A & B).

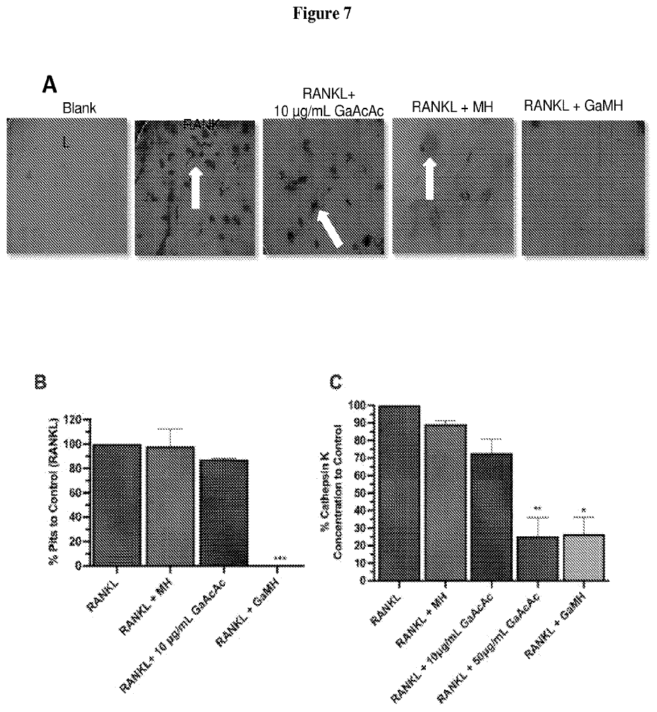

Further assessments using bone resorption assays were performed by differentiating pre-OC cells (RAW 264.7) & mHSCs into OCs on a bone matrix (bovine cortical bone slices) in an ex vivo setting. After OC differentiation, the bone slices were initially stained with TRAP and later with 0.5% toluidine blue stain to assess the pits formed by activated OC upon bone matrix degradation (FIG. 7A). With the treatment with 10 μg/mL GaAcAc solution, the resorption pits visible in positive control cells, were slightly reduced (FIG. 7A; p>0.05). Treatment with GaMH (containing 10 μg/mL) during OC differentiation resulted in lack of visible resorption pits on bone slices (FIG. 7B; p<0.0001) which agrees with the observation on bone resorption assay using OCs from mHSCs.

During the pit resorption assay, cell media was aliquoted after OC activation and assessed for CTSK marker using ELISA. MH treatment did not affect levels of CTSK after OC activation from RAW 264.7 cells while treatment with GaAcAc solution (10 g/mL) caused a slight reduction (1.4 times reduction) in CTSK concentration (FIG. 7C). The level of CTSK was markedly reduced (4 times reduction) upon treatment with 50 g/mL GaAcAc solution or GaMH (containing GaAcAc at 10 μg/mL) (FIG. 7C; p>0.001). From additional studies on differentiation of mHSCs to OCs on bone slices, we observed a pronounced reduction in CTSK levels with GaMH treatment compared to GaAcAc solution.

### In Vivo Characterization of GaMH

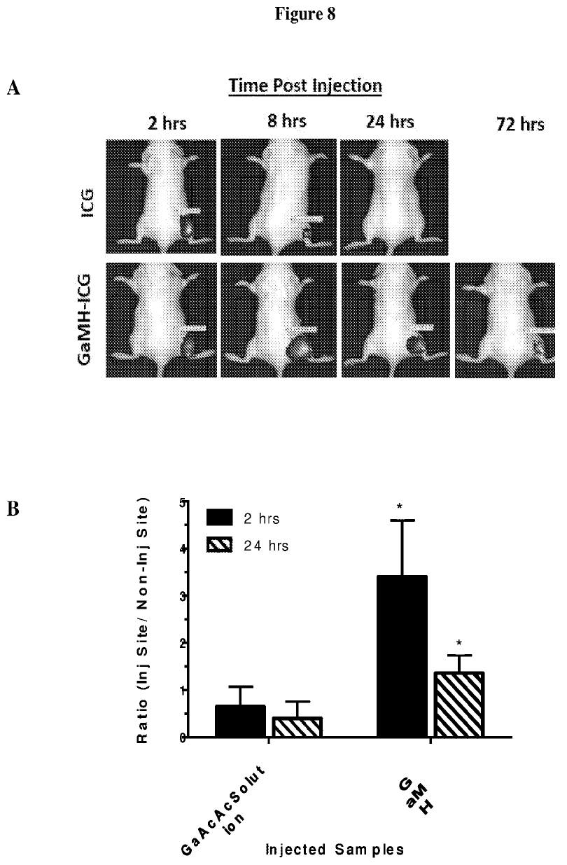

For the bioretention studies, GaMH was fluorescently labeled with ICG (ICG-GaMH) and administered by a single periosseous injection to the lower right hindlimb of BALB/c mice. Similarly, aqueous solutions of PBS (control) or ICG were administered. All mice were imaged via IVIS at various time points (FIG. 8). The fluorescent intensity from mice administered with aqueous solutions of ICG diminished by 8 hrs post-injection and completely disappeared by the time we acquired the images at 24 hrs post injection. In comparison, the fluorescence, from ICG-GaMH, was retained throughout the image acquisition at 72 hrs (FIG. 8). We conducted a follow up study involving injecting (a single periosseous injection) an equal dose of GaAcAc either as GaAcAc solution or GaMH. Mice were terminated at 2 hrs and 24 hrs post-injection. The concentration of Ga in each tissue sample was measured by ICP. The ratio of concentration of Ga at the injection site versus a non-injection site was obtained. At 2 hrs post-injection, the ratio of Ga retained at the injection site with GaMH was 5 times higher than when GaAcAc solution was administered. At 24 hrs post-injection, the ratio of Ga retention was 3.4 times higher with GaMH than with GaAcAc solution (FIG. 8B).

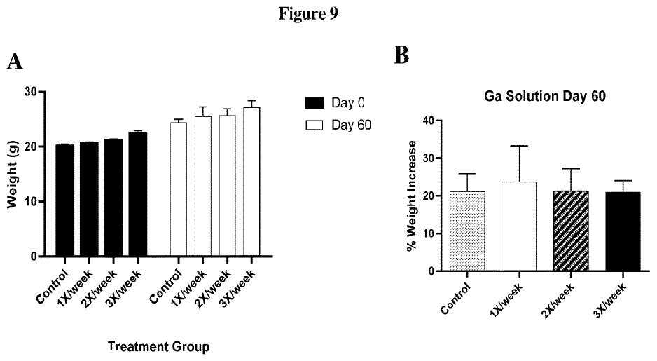

FIG. 9 (A & B) provide graphs and images showing that solutions of GaAcAc injected into BlkC57 mice did not show detectable toxicity based on weight of animals.

### Discussion

Bone resorptive disorders have posed a major health risk in most of the population, especially geriatrics. The detrimental effects of these disorders originate from weakened skeletal structures leading to fractures and delayed fracture healing. The pathological basis can be attributed to an imbalance between OC and OB regulation, specifically, an enhanced OC differentiation and function that will ultimately favor bone resorption. Feng, X. and J. M. McDonald, Annual review of pathology, 6: p. 121(2011 ). Hence, strategies that target OC differentiation and resorption (such as anti-resorptive treatment) without affecting OB functioning will be beneficial in treating bone resorptive disorders. Additionally, treatment regimens that mitigate off-target effects are more favorable and may have clinical utility. Thus, the need to explore alternative therapeutic approaches that maximize safety and efficacy remains at the forefront of drug development.

In this regard, earlier studies have shown that gallium nitrate (GaN) can effectively inhibit OC differentiation and function without affecting OBs. Verron et al., Biochem Pharmacol, 83(5): p. 671-9 (2012). In this work, we investigated a new gallium compound, GaAcAc. When comparing GaN and GaAcAc, though they share the same parent gallium ion, the varying ligands dictated changes in their physicochemical properties such as water solubility. GaN is highly water soluble, whereas GaAcAc, comprising of three molecules of acetylacetonate ligand group, is poorly water soluble. Greenwood, N., The chemistry of gallium, in Advances in Inorganic Chemistry and Radiochemistry. 1963, Elsevier. p. 91-134. As such, suitable delivery systems will be a necessity to realize the therapeutic potentials of gallium compounds by making it possible to deliver effective doses and overcome challenges associated with poor retention at the desired site and off-target distribution. Initial studies showed that GaAcAc is biocompatible with bone cells (pre-OCs and pre-OBs) at 10-50 μg/mL concentration range (FIG. 1). We also demonstrated the inhibitory effects of GaAcAc on OC differentiation processes (FIG. 2) which agrees with earlier studies with GaN. Verron et al., Biochemical Pharmacology, 83(5): p. 671-679 (2012).

With the goal of designing and developing a suitable delivery system for GaAcAc, we broadly screened a few delivery platforms and assessed the extent to which these will interfere with OC differentiation process before we eventually selected cellulose-derived thermosensitive gel (methylcellulose-based hydrogel, MH). MH consists of a well-knit mesh-like framework formed by the methoxyl groups (Thirumala et al., Cells, 2(3): p. 460-475 (2013)), which can entrap hydrophobic drugs such as GaAcAc, and release them in a controlled manner. The principle behind the solution-to-gel (sol-gel) phase transition of this unique cellulose-based hydrogel is due to the temperature-dependent fluctuating water molecules encasing the hydrophobic methoxyl groups. As the temperature dips below physiological temperature, the water molecules remain intact, leaving the gel in a solution state. In contrast, an increase in temperature releases the water molecules, favoring the solid-state, gel. Contessi et al., Cells, 2(3): p. 460-475 (2013).

We fabricated and characterized MH using the dispersion method at various concentrations of MC. The thermoresponsive property of the hydrogel as a drug delivery system facilitates ease of injection by remaining in solution phase at 25° C., while conversion to solid at the site of injection will favor bioretention and less off-target distribution. We applied rheological evaluation that explores changes in storage (G′) and loss (G″) moduli with increase in temperature. The storage modulus (G′) measures elasticity of the material and its ability to store energy, whereas the loss modulus (G″) is a measure of the ability to release energy. By measuring the G′ & G″ moduli over a temperature range, we were able to assess the structural state of the hydrogel at a different temperature, the sol-gel transition of the hydrogel, and the gelation temperature. The liquid behavior is associated with a G″ (loss of modulus) that is higher than G′ (storage modulus). At the gelation temperature and beyond, the storage modulus (G′) is greater than loss modulus (G″), which reflects a change to a gel phase. The pattern correlates with previous studies in the rheological assessment of the thermoreversible hydrogels. Tajvidi et al., Journal of Applied Polymer Science, 101(6): p. 4341-4349 (2005).

The rheological evaluation of hydrogels showed that at MC concentrations below 6% w/v there was no crossover between G′ and G″, indicating lack of thermoresponsive behavior. The moduli crossover, signaling gelation occurred in hydrogels prepared with MC concentration of 6%, however, gelation temperature was close to 50° C. (FIG. 3A). Therefore, hydrogels prepared at 8% w/v MC was selected for GaAcAc delivery because the gelation temperature of 37° C. is ideal for an injectable application. We were not able to obtain viable hydrogels at MC concentrations above 8% partly due to high viscosity of MC solution. Further assessment showed that hydrogels prepared with MC are biocompatible and undergo a gradual loss of the hydrogel layer (FIG. 3D). These characteristics agree with earlier reports that the internal framework of MC-based hydrogels can degrade over time. Thirumala et al., Cells, 2(3): p. 460-475 (2013).

Subsequent studies showed that a broad concentration range of GaAcAc (25-200 μg/mL) can be loaded onto the hydrogels while maintaining the thermoresponsive properties (FIG. 4A). The release kinetics of GaMH showed that GaAcAc was released in a controlled manner over time, comprised of an initial fast release phase that was followed by a slow-release phase (FIG. 4B). The trend suggests that GaAcAc encapsulated in the hydrogel was initially released due to its presence on the surface (burst release), followed by the gradual release that could be linked to continued degradation of the gel. Further elucidations are required to understand the complete mechanism behind GaAaAc release and its stability; however, our data shows that GaAcAc can be effectively loaded into a methylcellulose-based hydrogel (MH), and we are unaware of any other studies that have focused on delivery systems of gallium compounds for application in bone disorders.

The next parameter assessed the OC inhibitory effects of hydrogels loaded with GaAcAc (GaMH) in comparison to GaAcAc solution (not loaded in hydrogels). In our initial investigation, both GaAcAc alone and GaMH significantly inhibited OC differentiation (FIGS. 2 & 5). We were able to verify that blank hydrogels (MH) did not interfere with OC differentiation, which indicated that efficacy of GaMH could be attributed to GaAcAc loading. Overall, our findings suggest that hydrogels loaded with GaAcAc (GaMH) showed better efficacy in hindering OC differentiation when compared to GaAcAc solution. To further characterize the effect of GaAcAc and GaMH we performed western blotting analyses of key markers that are involved in OC differentiation and function in bone resorption. Previous studies have reported that complementary binding of RANKL to RANK receptor triggered a myriad signaling cascade involving the following markers; cFos, TRAF6, TRAP, and NFAT2. Blumer et al., Mechanisms of Development, 129(5): p. 162-176 (2012). Increasing the concentration of GaAcAc from 10 μg/mL to 50 μg/mL resulted in marked downregulation of all the expression of all the identified OC differentiation markers (FIG. 6). It is noteworthy that GaMH containing 10 μg/mL was superior to GaAcAc solution at the same concentration when considering the expression levels of OC differentiation markers (FIG. 6).

To further assess the effect of GaAcAc solution and GaMH on OC function, we performed resorption studies (ex-vivo) using bovine bone slices (FIG. 7). In the study, we observed the extent to which differentiated OCs will resorb bone slices. Upon staining, the pits on the bone slices were visually clearer and facilitated a quantitative analysis of treatment with GaAcAc versus GaMH. Treatment with between blank (no cells on chip), positive control, and treatment groups, respectively. Wells treated with MH were unaffected; however, there was a slight decrease in the pits formed in wells treated with 10 μg/mL GaAcAc. Treatments with GaAcAc (50 μg/mL) and GaMH were found to significantly decrease the number of pits formed due to the inhibition of the activated osteoclasts compared to a positive control (FIG. 7B). This OC inhibitory trend of GaMH in vitro and ex vivo coincides with the trend observed initially on GaAcAc (50 μg/mL) treatment against differentiated OCs (FIG. 7A).

To further confirm the anti-resorptive trend, the concentration of CTSK release during the resorptive process was analyzed. Bone resorption involves synthesis of CTSK by OCs that is subsequently secreted to degrade the organic matrix. Normally, the release of CTSK is directly proportional to OC resorption as a viable marker of the extent of OC function. Song et al., 233(12): p. 9674-9684 (2018). In which case, decrease in the levels of secreted CTSK will correspond to inhibition of bone resorption. Reduction in levels of CTSK was observed in treatments with GaAcAc solution (10 μg/mL and 50 g/mL) in a dose dependent manner. Our observation showed that GaMH (containing GaAcAc 10 μg/mL) performed better than GaAcAc solution at the same concentration level (FIG. 7C) in reducing CTSK concentration, which corresponds with inhibiting OC function. The performance of GaMH compared to GaAcAc solution could be attributed to the ability to control the release of GaAcAc and maximize its availability and efficacy. We are also of the opinion that the thermoresponsive behavior of the hydrogel will be closely linked to the bioretention property and the ability to achieve localized therapeutic effect, and maximize the effect at the desired site while limiting off-target effects. Our initial assessment demonstrated the bio-retention of GaMH following perisseous injection in BALB/c mice as measured by close monitoring of injection site for fluorescence and concentration of gallium via ICP.

In conclusion, we assessed the potential of a new gallium compound (GaAcAc) as an anti-resorptive therapeutic agent. The stability and thermoresponsive behavior of MC-based hydrogel delivery systems were demonstrated. MC-based hydrogels showed attractive qualities in GaAcAc delivery such as (a) GaAcAc loading onto the hydrogels did not interfere with the thermoreponsive behavior, (b) the release of GaAcAc occurred in a controlled manner to maximize exposure with OCs, (c) based on expression of key markers, GaAcAc released from hydrogels was effective in inhibiting OC differentiation and function, (d) hydrogels loaded with GaAcAc (GaMH) showed superior performance in inhibiting OC differentiation and function compared to GaAcAc solution, and (e) GaMH demonstrated ability to achieve longer bio-retention at the site of injection compared to GaAcAc solution, which could maximize exposure at site of action while minimizing off-target distribution.

### Example 2

### Polymersomes and Additional Hydrogel Compositions

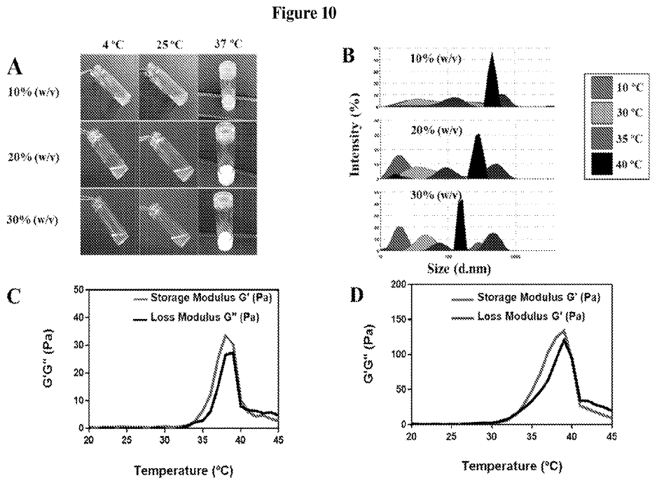

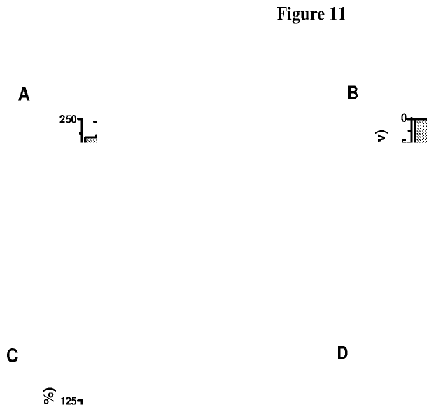

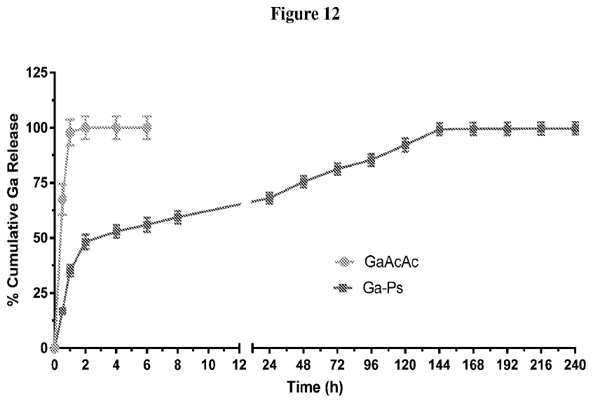

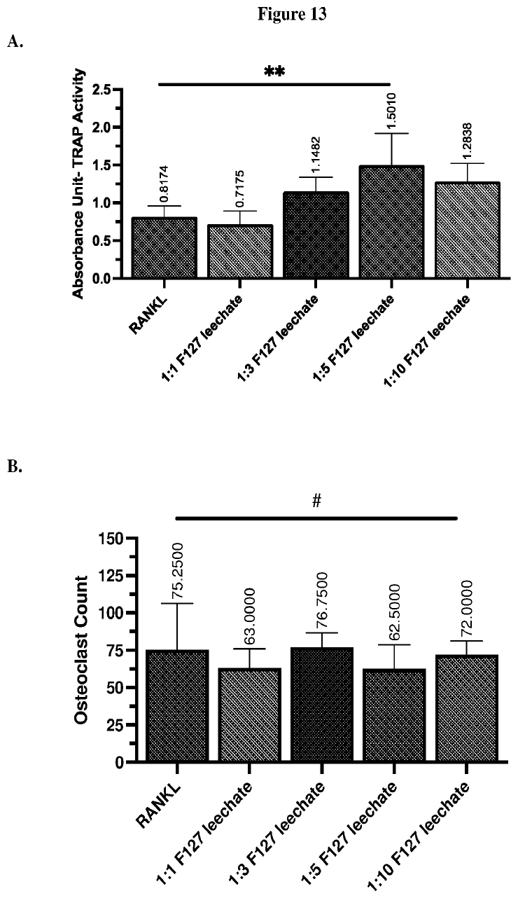

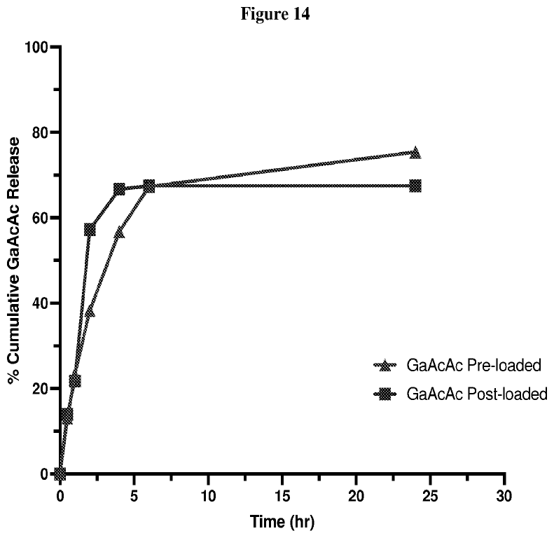

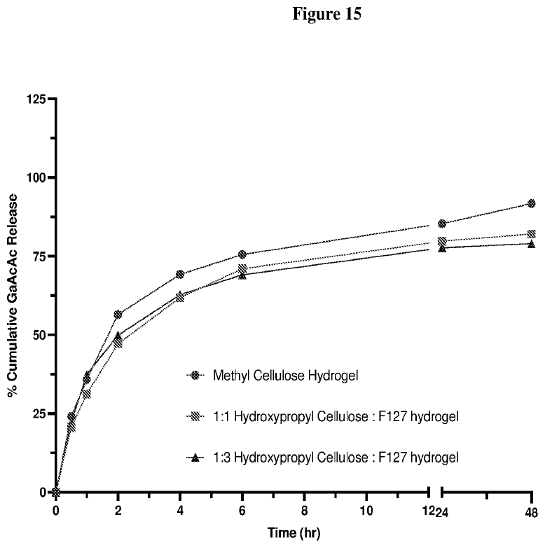

Additional experiments were carried out to demonstrate that other thermoresponsive hydrogels such as PLA-b-PEG-PLA could be used to deliver gallium compounds. FIGS. 10A-10D show the characterization of thermoresponsive behavior of the PLA-b-PEG-b-PLA hydrogel. The inventors also loaded gallium compounds into polymersomes to help increase the retention and provide sustained release of the gallium compounds. FIGS. 11A-11D show the characterization of polymersomes Ga-Ps. FIG. 12 shows the in-vitro release of Ga from polymersomes Ga-Ps in PBS. FIGS. 13A & 13B show leachates from hydrogels prepared using Pluronic F127 did not affect osteoclast differentiation. FIG. 14 shows the cumulative GaAcAc release from F127 Hydrogel. Finally, FIG. 15 shows the cumulative GaAcAc release from different hydrogel formulations.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A composition for inhibiting bone resorption, comprising a gallium compound and a thermoresponsive hydrogel.

2. The composition of claim 1, wherein the gallium compound is selected from the group consisting of gallium acetylacetonate, gallium nitrate, and gallium citrate, gallium maltolate, gallium carbonate, gallium acetate, gallium triacetate, gallium tartrate, gallium oxide, gallium hydroxide, and gallium hydrated oxide.

3. The composition of claim 1, wherein the gallium compound is gallium acetylacetonate.

4. The composition of claim 1, wherein the thermoresponsive hydrogel is a cellulose-based hydrogel.

5. The composition of claim 4, wherein the thermoresponsive hydrogel is a methylcellulose hydrogel.

6. The composition of claim 1, wherein the thermoresponsive hydrogel is a natural polymer-based hydrogel.

7. The composition of claim 1, wherein the thermoresponsive hydrogel is a PLA-b-PEG-b-PLA hydrogel.

8. The composition of claim 1 further comprising polymersomes.

9. The composition of claim 8, wherein the polymersomes are PLA-PEG polymersomes.

10. The composition of claim 1, wherein the thermoresponsive hydrogel comprises a bone-seeking ligand.

11. The composition of claim 1, wherein the thermoresponsive hydrogel comprises a bone-retentive ligand.

12. The composition of claim 1, wherein the composition comprises a scaffold.

13. A method of inhibiting bone resorption in a subject, comprising administering a therapeutically effective amount of composition comprising a gallium compound and a thermoresponsive hydrogel to a subject in need thereof.

14. The method of claim 13, wherein the subject has been diagnosed as having a bone growth disease or disorder.

15. The method of claim 14, wherein the subject has been diagnosed as having bone growth disease or disorder selected from the group consisting of osteogenesis imperfecta, disorders caused by increased osteoclastogenesis or bone loss associated with inflammatory conditions, infection, genetic and age-related bone disorders such as osteoporosis, osteopenia, Paget's disease, metastatic bone cancer, myeloma bone disease, bone fracture healing, and bone graft repair.

16. The method of claim 13, wherein the subject has been diagnosed as having an increased risk of developing a bone growth disease or disorder.

17. The method of claim 13, wherein the gallium compound is selected from the group consisting of gallium acetylacetonate, gallium nitrate, and gallium citrate.

18. The method of claim 13, wherein the gallium compound of the composition is gallium acetylacetonate.

19. The method of claim 13, wherein the thermoresponsive hydrogel of the composition is a methylcellulose hydrogel.

20. The method of claim 13, wherein the thermoresponsive hydrogel comprises a bone-seeking ligand.
